(19) United States
(12) Patent Application Publication
(10) Pub. No.: US 2024/0180616 A1
(43) Pub. Date: Jun. 6, 2024

(54) ANNOTATION FOR ELECTROPORATION ABLATION

(71) Applicant: Boston Scientific Scimed, Inc., Maple Grove, MN (US)

(72) Inventors: Kara Elizabeth Garrott, Minneapolis, MN (US); Brendan Early Koop, Ham Lake, MN (US); Eric Daniel Buck, Shoreview, MN (US); Amanda Christine Kingston, Waltham, MA (US)

(21) Appl. No.: 18/510,087

(22) Filed: Nov. 15, 2023

Related U.S. Application Data
(60) Provisional application No. 63/429,388, filed on Dec. 1, 2022.

(51) Int. Cl. A61B 18/14

(52) U.S. Cl. CPC A61B 18/1492; A61B 2018/00577

(57) ABSTRACT

A system for performing electroporation ablation of target tissue in a chamber of a patient's heart is disclosed. The system including a catheter including an electrode assembly having a plurality of electrodes, wherein the catheter is adapted to position the electrode assembly at a plurality of locations proximate the target tissue, a graphical display, and a controller. The controller configured to generate, on the graphical display, a graphical representation of the electrode assembly. The controller configured to generate, on the graphical display, a graphical representation of a model of electric fields generated in response to delivery of pulsed electrical signals to selected ones of the plurality of electrodes.

### CROSS-REFERENCE TO RELATED APPLICATIONS

This application claims the benefit of U.S. Provisional Application No. 63/429,388, entitled “ANNOTATION FOR ELECTROPORATION ABLATION,” and filed Dec. 1, 2022, which is hereby incorporated by reference in its entirety.

### TECHNICAL FIELD

The present disclosure relates generally to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques can indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells. Planning irreversible electroporation ablation procedures can be difficult due to the lack of acute visualization or data indicating which tissues have been irreversibly electroporated compared to tissues that have been reversibly electroporated.

### SUMMARY

In an Example 1, a system for performing electroporation ablation of target tissue in a chamber of a patient's heart, the system including a catheter including an electrode assembly having a plurality of electrodes, wherein the catheter is adapted to position the electrode assembly at a plurality of locations proximate the target tissue, a graphical display, and a controller. The controller configured to generate, on the graphical display, a graphical representation of the electrode assembly. The controller configured to generate, on the graphical display, a graphical representation of a model of electric fields generated in response to delivery of pulsed electrical signals to selected ones of the plurality of electrodes. Prior to delivery of the pulsed electrical signals to the selected ones of the plurality of electrodes at each of the plurality of locations, the controller configured to generate, on an anatomical map of the heart on the graphical display, a predicted lesion zone corresponding to an intersection of the model of electric fields and a surface of the anatomical map. After or concurrently with delivery of the pulsed electrical signals to the selected ones of the plurality of electrodes at each of the plurality of locations, the controller configured to automatically annotate the anatomical map on the graphical display by applying an ablation marker based on the predicted lesion zone corresponding to each of the plurality of locations.

In an Example 2, the system of Example 1, wherein at each of the plurality of locations, the controller is further configured to generate, on the anatomical map on the graphical display, a first overlap zone defined by an area of overlap of the corresponding ablation marker and one previously applied ablation marker.

In an Example 3, the system of Example 2, wherein at each of the plurality of locations, the controller is further configured to generate, on the anatomical map on the graphical display, a second overlap zone defined by an area of overlap of the corresponding ablation marker and two or more previously applied ablation markers.

In an Example 4, the system of Example 3, wherein the ablation marker, the first overlap zone and the second overlap zone each have a different visual appearance on the anatomical map.

In an Example 5, the system of any of Examples 2-4, wherein the controller is further configured to automatically annotate the anatomical map on the graphical display to identify each ablation marker that overlaps with at least two different ablation markers.

In an Example 6, the system of Example 5, wherein the controller is further configured to automatically annotate the anatomical map on the graphical display to display an outer boundary of a series of ablation markers that each overlap with at least two spatially-adjacent ablation markers.

In an Example 7, the system of any of Examples 1-6, wherein the controller is further configured to automatically identify a gap between any two spatially-adjacent ablation markers that overlap with at least two different ablation markers.

In an Example 8, the system of any of Examples 1-7, wherein each predicted lesion zone and the corresponding ablation marker have substantially the same geometry.

In an Example 9, the system of any of Examples 1-8, wherein each predicted lesion zone and the corresponding ablation marker have substantially the same visual appearance.

In an Example 10, the system of any of Examples 1-7, wherein each predicted lesion zone and the corresponding ablation marker have different visual appearances.

In an Example 11, the system of any of Examples 1-10, wherein a visual appearance of the predicted lesion zones or the ablation markers varies as a function of one or more ablation parameters.

In an Example 12, the system of any of Examples 2-11, wherein each first overlap zone has a different visual appearance than each second overlap zone.

In an Example 13, the system of any of Examples 1-12, wherein the catheter is configured for selective delivery of monopolar and bipolar ablative energy.

In an Example 14, the system of any of Examples 1-13, wherein the system is included in one of an electroporation catheter system or an electro-anatomical mapping system.

In an Example 15, the system of any of Examples 1-14, wherein the anatomical map is an electroanatomical map.

In an Example 16, a system for performing electroporation ablation of target tissue in a chamber of a patient's heart, the system including a catheter including an electrode assembly having a plurality of electrodes, wherein the catheter is adapted to position the electrode assembly at a first location proximate the target tissue, a graphical display, and a controller. Prior to delivery of ablative energy to the plurality of electrodes, the controller configured to generate, on the graphical display, a graphical representation of a model of electric fields generated by the plurality of electrodes. Also prior to delivery of ablative energy to the plurality of electrodes, the controller configured to generate, on an anatomical map of the heart on the graphical display, a first predicted lesion marker corresponding to an intersection of the model of electric fields and a surface of the anatomical map when the electrode assembly is at a first position proximate the target tissue. After or concurrently with delivery of ablative energy to the plurality of electrodes, the controller configured to automatically annotate the anatomical map on the graphical display with a first ablation marker corresponding to the first predicted lesion marker.

In an Example 17, the system of Example 16, wherein the controller is further configured to, when the electrode assembly is at a second location proximate the target tissue and after automatically annotating the anatomical map with the first ablation marker, generate, on the atomical map of the heart on the graphical display, a second predicted lesion marker corresponding to the intersection of the model of electric fields and the surface of the anatomical map prior to delivery of the ablative energy to the plurality of electrodes.

In an Example 18, the system of Example 17, wherein the controller is further configured to, after or concurrently with delivery of the ablative energy to the plurality of electrodes when the electrode assembly is at the second position proximate the target tissue, automatically annotate the anatomical map on the graphical display with a second ablation marker corresponding to the second predicted ablation marker.

In an Example 19, the system of Example 17, wherein the controller is further configured to, when the electrode assembly is at the second position proximate the target tissue and after automatically annotating the anatomical map with the first ablation marker, generate, on the atomical map of the heart on the graphical display, a third predicted lesion marker defined by an area of overlap of the first ablation marker and the second predicted lesion marker.

In an Example 20, the system of Example 19, wherein the controller is further configured to, after or concurrently with delivery of the ablative energy to the plurality of electrodes when the electrode assembly is at the second position proximate the target tissue, automatically annotate the anatomical map on the graphical display with a third ablation marker corresponding to the third predicted ablation marker.

In an Example 21, the system of Example 20, wherein the third ablation marker has a different visual appearance than the first and second ablation markers.

In an Example 22, the system of Example 16, wherein the first predicted lesion marker and the first ablation marker each have a different visual appearance.

In an Example 23, the System of Example 16, wherein the catheter is configured for selective delivery of monopolar and bipolar ablative energy, and wherein the controller is configured to generate the model of the electric fields when the catheter configured for delivery of monopolar ablative energy differently than when the catheter is configured for delivery of bipolar ablative energy.

In an Example 24, a system for performing electroporation ablation of target tissue in a chamber of a patient's heart, the system including a catheter including an electrode assembly having a plurality of electrodes, wherein the catheter is adapted to position the electrode assembly at a location proximate an ablated region of the target tissue, a graphical display, and a controller. The controller configured to generate, on the graphical display, a graphical representation of the electrode assembly and a first ablation marker corresponding with the ablated region. The controller configured to generate, on the graphical display, a graphical representation of a model of electric fields generated in response to delivery of pulsed electrical signals to selected ones of the plurality of electrodes. Prior to delivery of the pulsed electrical signals to the selected ones of the plurality of electrodes at the location, the controller configured to generate, on an anatomical map of the heart on the graphical display, a predicted lesion zone corresponding to an intersection of the model of electric fields and a surface of the anatomical map and an overlap zone corresponding to an intersection of the predicted lesion zone and the first ablation marker. After or concurrently with delivery of the pulsed electrical signals to the selected ones of the plurality of electrodes at the location, the controller configured to automatically annotate the anatomical map on the graphical display by applying a second ablation marker based on the predicted lesion zone corresponding to the location and by defining the overlap zone.

In an Example 25, the system of Example 24, wherein the controller is further configured to automatically identify on the anatomical map on the graphical display another overlap zone spatially adjacent to the overlap zone.

In an Example 26, the system of Example 24, wherein the controller is further configured to automatically annotate on the graphical display a first contiguous string of spatially-adjacent overlap zones including the overlap zone.

In an Example 27, the system of Example 26, wherein the controller is further configured to automatically annotate on the graphical display a second contiguous string of spatially-adjacent overlap zones spaced-apart on the target tissue from the first contiguous string of spatially-adjacent overlap zones.

In an Example 28, the system of Example 27, wherein the controller is further configured to automatically identify on the graphical display a gap on the target tissue disposed between the first contiguous string of spatially-adjacent overlap zones spaced-apart and the second contiguous string of spatially-adjacent overlap zones.

In an Example 29, the system of Example 28, wherein the controller is further configured to automatically highlight on the graphical display the gab based on distance between the first contiguous string of spatially-adjacent overlap zones spaced-apart and the second contiguous string of spatially-adjacent overlap zones.

In Example 30, a process for use with electroporation ablation of target tissue in a chamber of a patient's heart with a catheter including an electrode assembly having a plurality of electrodes, wherein the catheter is adapted to position the electrode assembly at a plurality of locations proximate the target tissue. The process including generating, on a graphical display, a graphical representation of the electrode assembly, generating, on the graphical display, a graphical representation of a model of electric fields generated in response to delivery of pulsed electrical signals to selected ones of the plurality of electrodes, prior to delivery of the pulsed electrical signals to the selected ones of the plurality of electrodes at each of the plurality of locations, generating, on an anatomical map of the heart on the graphical display, a predicted lesion zone corresponding to an intersection of the model of electric fields and a surface of the anatomical map, and after or concurrently with delivery of the pulsed electrical signals to the selected ones of the plurality of electrodes at each of the plurality of locations, automatically annotating the anatomical map on the graphical display by applying an ablation marker based on the predicted lesion zone corresponding to each of the plurality of locations.

In Example 31, the process of Example 30, and further generating, on the anatomical map on the graphical display, a first overlap zone defined by an area of overlap of the corresponding ablation marker and one previously applied ablation marker at each of the plurality of locations.

In Example 32, the process of Example 31, and further generating, on the anatomical map on the graphical display, a second overlap zone defined by an area of overlap of the corresponding ablation marker and two or more previously applied ablation markers at each of the plurality of locations.

In Example 33, the process of Example 31, and further automatically annotating the anatomical map on the graphical display to identify each ablation marker that overlaps with at least two different ablation markers.

In Example 34, the process of Example 30, and further automatically identifying on the anatomical map on the graphical display a lesion line of contiguous series of spatially-adjacent ablation markers including the ablation marker and previously applied ablation markers.

In Example 35, the process of Example 30, wherein the catheter is configured for selective delivery of monopolar and bipolar ablative energy, generating indicia in the model of the electric fields on the graphical display when the catheter configured for delivery of monopolar ablative energy differently than when the catheter is configured for delivery of bipolar ablative energy.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an example clinical setting for treating a patient, and for treating a heart of the patient, the example clinical setting having an example electrophysiology system.

FIG. 2 is a block diagram illustrating an example controller for use with the example electrophysiology system of FIG. 1.

FIG. 3 is a flow diagram illustrating an example configuration of the example controller of FIG. 2.

FIG. 4A is a schematic diagram illustrating an example graphical representation, such as a visualization on a display of the electrophysiology system of FIG. 1 as can be configured by the example controller of FIG. 2, of a surface of an anatomical map of the heart intersecting with a model of an electric field of an example electroporation catheter configured in a first, or monopolar mode and a predicted lesion zone.

FIG. 4B is a schematic diagram illustrating an example graphical representation, such as a visualization on the display of an electrophysiology system of FIG. 1 as can be configured by the example controller of FIG. 2, including an ablation marker annotating the surface of the anatomical map of the heart based on the predicted lesion zone as illustrated in FIG. 4A.

FIG. 4C is a schematic diagram illustrating an example graphical representation, such as a visualization on a display of the electrophysiology system of FIG. 1 as can be configured by the example controller of FIG. 2, of the surface of an anatomical map of the heart including the ablation marker of FIG. 4B intersecting with a model of another electric field of the example electroporation catheter configured in a first, or monopolar, mode and a second predicted lesion zone.

FIG. 4D is a schematic diagram illustrating an example graphical representation, such as a visualization on the display of an electrophysiology system of FIG. 1 as can be configured by the example controller of FIG. 2, including a second ablation marker annotating the surface of the anatomical map of the heart based on the second predicted lesion zone as illustrated in FIG. 4C and a first overlap zone defined by an area of overlap of the second ablation marker and the previously applied ablation marker.

FIG. 5A is a schematic diagram illustrating an example graphical representation, such as visualization on a display of the electrophysiology system of FIG. 1 as can be configured by the example controller of FIG. 2, of a surface of an anatomical map of the heart intersecting with a model of an electric field of an example electroporation catheter configured in a second, or bipolar, mode.

FIG. 5B is schematic diagram illustrating an example graphical representation, such as a visualization on the display of an electrophysiology system of FIG. 1 as can be configured by the example controller of FIG. 2, an ablation marker annotating the surface of the anatomical map of the heart based on the predicted lesion zone as illustrated in FIG. 5A

FIG. 6A is schematic diagram illustrating an example graphical representation, such as a visualization on the display of an electrophysiology system of FIG. 1 as can be configured by the example controller of FIG. 2, including a plurality of ablation markers annotating the surface of the anatomical map of the heart arranged to include a plurality of overlapping zones.

FIG. 6B is schematic diagram illustrating an example graphical representation, such as a visualization on the display of an electrophysiology system of FIG. 1 as can be configured by the example controller of FIG. 2, including a plurality of ablation markers annotating the surface of the anatomical map of the heart arranged to include a plurality of overlapping zones of FIG. 6A and including an outer boundary of a series of ablation markers that each overlap with at least two spatially-adjacent ablation markers and identifying a gap between any two spatially-adjacent ablation markers that overlap with at least two different ablation markers.

While the disclosure is amenable to various modifications and alternative forms, specific embodiments have been shown by way of example in the drawings and are described in detail below. The intention, however, is not to limit the disclosure to the particular embodiments described. Rather, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

Throughout the present disclosure including the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

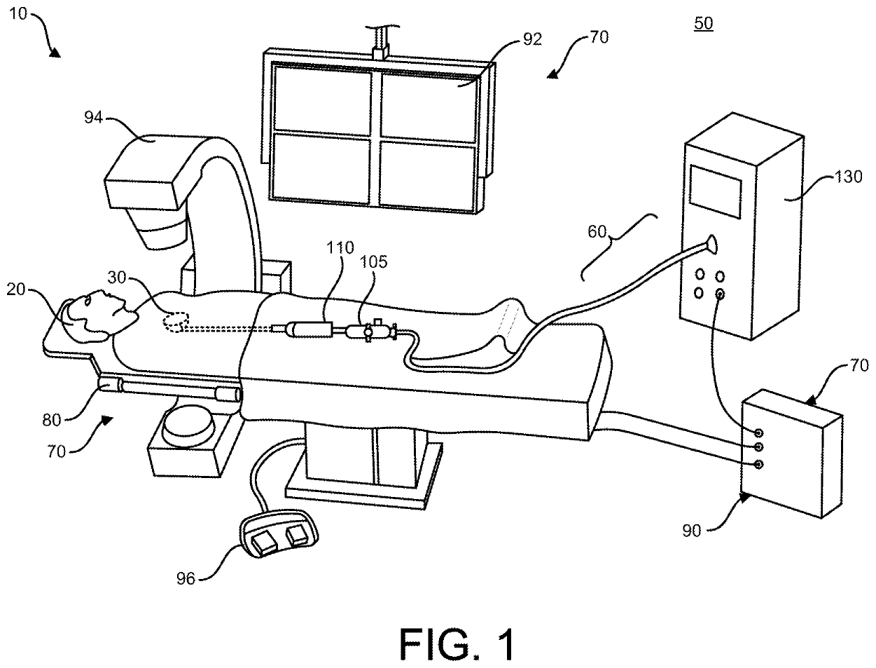

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1. Other arrangements of connecting elements, including wireless connecting elements, are contemplated.

The electroporation catheter system 60 is configured to deliver electric field energy to targeted tissue in the patient's heart 30 to create cell death in tissue, for example, rendering the tissue incapable of conducting electrical signals. Also, the electroporation catheter system 60 is configured to generate, based on models of electric fields, graphical representations of the electric fields that can be produced using the electroporation catheter 105 and to overlay, on the display 92, the graphical representations of the electric fields or expected or predicted lesions on an anatomical map of the patient's heart to aid a user in planning ablation by irreversible electroporation using the electroporation catheter 105 prior to delivering energy. In embodiments, the electroporation catheter system 60 is configured to generate the graphical representations of the electric fields based on characteristics of the electroporation catheter 105 and the position of the electroporation catheter 105 in the patient 20, such as in the heart 30 of the patient 20. The electroporation catheter system 60 is configured to generate the graphical representations of the electric fields based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end configured to be deployed proximate target tissue, such as within a chamber of the patient's heart. The distal end may include a basket, balloon, spline, configured tip, or other electrode deployment mechanism to effect treatment. The electrode deployment mechanism includes an electrode assembly, or array, comprising of an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, to effect electroporation.

A selected electrical field can be generated with the electrodes to effect electroporation. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly.

The electroporation console 130 is configured to control aspects of the electroporation catheter system 60. In embodiments, the electroporation console 130 is configured to provide one or more of the following: modeling the electric fields that can be generated by the electroporation catheter 105, which often includes consideration of the physical characteristics of the electroporation catheter 105 including the electrodes and spatial relationships of the electrodes on the electroporation catheter 105 and, whether the electroporation catheter 105 is in bipolar or monopolar mode; generating the graphical representations of the electric fields, which often includes consideration of the position of the electroporation catheter 105 in the patient 20 and characteristics of the surrounding tissue; and overlaying, on the display 92, the generated graphical representations on an anatomical map. In some examples, the electroporation control console 130 is configured to generate the anatomical map. In some examples, the EAM system 70 is configured to generate the anatomical map for display on the display 92.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an anatomical map of the patient and, in some instances, on an electro-anatomical map of the patient's heart. The integrated system can include the capability to enhance the efficiency of clinical workflows, including enhancement of providing a visual representation to the clinician of ablation lesions of portions of the patient's heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

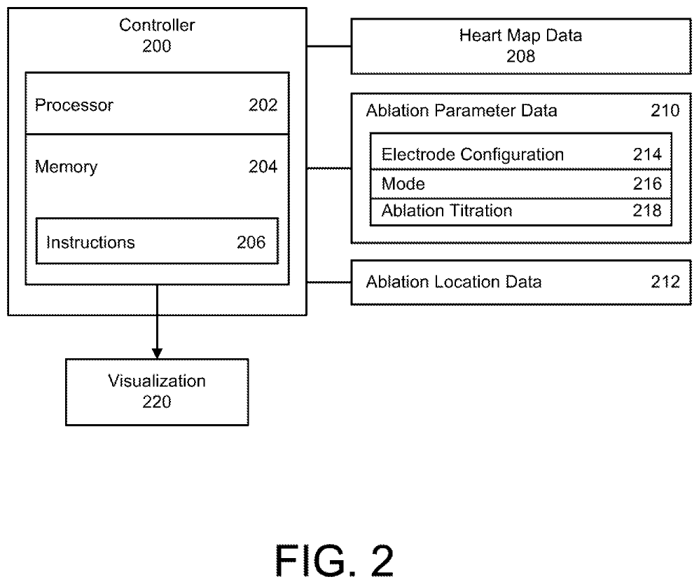

FIG. 2 illustrates an example controller 200 that can be used with the example electrophysiology system 50, such as a controller of the example electroporation catheter system 60, which may include a controller of the electroporation console 130, a controller of the example EAM system 70, which may include a mapping and navigation controller 90, a controller of an integrated electroporation catheter system 60 and EAM system 70, or a controller for use with the electroporation catheter system 60 and EAM system 70. The controller 200 can be implemented to provide an ablation lesion visualization for irreversible electroporation in the example. In some examples, the controller 200 can also be implemented to provide for selected annotations in regions not subjected to irreversible electroporation. The controller 200 can include a processor 202 and a memory 204. The memory 204 stores processor executable instructions 206. In one example, the processor executable instructions can be in the form of a program, such as a computer program or application. The processor 202 can execute the instructions 206 that can be included in configuring the controller 200. In one example, the controller 200 can be implemented to include a computing device such as a laptop computer, a workstation, a desktop computer, a tablet, or a smartphone. In such examples, the controller 200 can include additional components such as a display, a touchscreen, speakers or other output devices, a keyboard or other input devices, or communication circuitry such as computer network adapters. The controller 200 may be implemented in a variety of architectures and components, such as the processor 202 and memory 204, may be distributed in various locations.

In one example, the processor 202 may include a plurality of main processing cores to run an operating system and perform general-purpose tasks on an integrated circuit. The processor 202 may also include built-in logic or a programmable functional unit, also on the same integrated circuit with a heterogeneous instruction-set architecture. In additional to multiple general-purpose, main processing cores and the application processing unit, controller 200 can include other devices or circuits such as graphics processing units or neural network processing units, which may include heterogeneous or homogenous instruction set architectures with the main processing cores. For example, the controller 200 may be used to perform other tasks such as in the case of a computing device including the resonance sound amplification device.

Memory 204 is an example of computer storage media. Computer storage media includes RAM, ROM, EEPROM, flash memory or other memory technology, CD-ROM, digital versatile discs (DVD) or other optical storage, magnetic cassettes, magnetic tape, magnetic disk storage or other magnetic storage devices, USB flash drive, flash memory card, or other flash storage devices, or other storage medium that can be used to store the desired information and that can be accessed by the processor 202. Any such computer storage media may be part of the controller 200 and implemented as memory 204. Memory 204 is a non-transitory, processor readable memory device. Accordingly, a propagating signal by itself does not qualify as storage media or memory 204.

The controller 200 may be configured to receive inputs or information from the electrophysiology system 50, such as inputs from the electroporation catheter system 60 and EAM system 70 including the electroporation console 130 and the mapping and navigation controller 90, for storage in memory 204 and use by the instructions 206. For example, the controller 200 can receive an input representative of the anatomical map of the heart, or heart map data 208, which heart map data 208 can include the data regarding representations of the geometric anatomical map of the heart and the electro-anatomical map of the heart, such as from the EAM system 70. Additionally, the controller 200 can receive an ablation parameter data 210 and an ablation location data 212, such as from the electroporation catheter system 60. In some examples, the anatomical heart map data 208, the ablation parameter data 210, or the ablation location data 212, can be generated by the controller 200 to be stored in the memory 204 or accessed by the instructions 206. In one example, the ablation parameter data 210 can include electrode configuration data 214 about electrode configuration on the electroporation catheter 105, including which electrodes on the electroporation catheter 105 are to be activated and in which polarity (anode or cathode), electrode mode data 216 regarding the mode of the electroporation catheter 105 including bipolar mode or monopolar mode and the electric field vector generated with the electroporation catheter 105, and ablation titration data 218 regarding information about the pulsed field ablation including pulse energy strength, pulse time, whether the pulse is biphasic or monophasic, and other information regarding the ablation as determinable from the electroporation catheter system 60. For instance, the ablation parameter data 210 can be provided from the electroporation console 130 based on measurements, settings, or configurations of the electroporation catheter system 60.

The controller 200 is configured to generate a visualization 220 that can include determined predicted, or anticipated, effects of therapy with reference to the anatomical map of the heart, such anticipated lesion effects based on settings of the electroporation catheter system 60 prior to ablation, and provide ablation markers illustrating lesion location with reference to the anatomical map of the heart that include lesion size and other information regarding the ablation performed with the electroporation catheter system 60. In one example, the controller 200 is configured to generate a visualization of overlapping zones of ablation markers in which two or more ablation markers include a same region of target tissue.

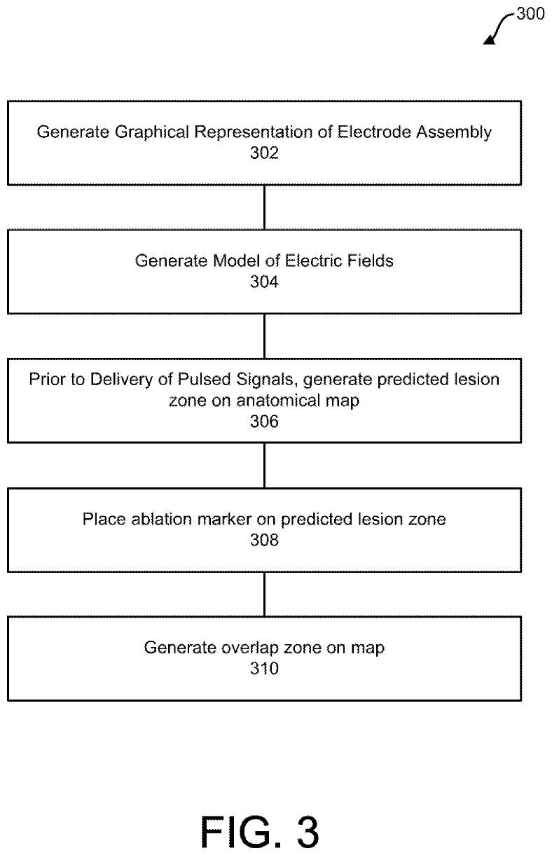

FIG. 3 illustrates a process 300 of configuring a controller, such as controller 200, while performing an electroporation of target tissue, such as in a chamber of a patient's heart. In one example, the controller is implemented as part of the EAM system 70 and operably coupled to the electroporation catheter system 60. Process 300 includes configuring the controller to generate a graphical representation of the electrode assembly at 302. For example, the graphical representation of the electrode assembly at 302 can include a graphical representation of the electroporation catheter 105 with respect to an anatomical map of the patient's heart. The graphical representation can include a schematic representation or other indicia that presents a model of the location of the electrode assembly of the electroporation catheter 105 with respect to the heart such as may be determined from the ablation location data 212, such as provided from the electroporation catheter system 60, applied to the anatomical heart map data 208, such as provided from the EAM system 70. Selected electrodes of the electrode assembly are configured to generate actual or predicted electric field within the heart as determined from the parameter data 210 such as provided from the electroporation catheter system 60. A graphical representation of a model of electric field is generated in response to delivery of pulsed electrical signals to selected electrodes of the plurality of electrodes at 304. The electric fields can change as the electrode assembly is moved with respect to the heart, and the graphical representation of the model of electric fields can change as well, such as in real-time. The graphical representation of the model of electric fields can include an area depicting the electric field having a strength to provide irreversible electroporation in the tissue of interest, an indicia in the representation of the of the model of the electric field indicating a mode of electrode array (such as whether the electrode array is configured to deliver monopolar energy or bipolar energy), as well as other indicators of aspects of the titration scheme, such as determined from ablation parameter data 210. In one example, indicia regarding field strength, mode, and titration scheme can include associated or corresponding colors and shades of colors that provide visual indication of the graphical representations.

Prior to delivery of the pulsed electrical signals to the selected ones of the plurality of electrodes at each of the plurality of locations, a predicted lesion zone corresponding to an intersection of the model of electric fields and a surface of the anatomical map is generated on the anatomical map of the heart on the graphical display at 306. The predicted lesion zones can be determined from the ablation parameter data 210 and ablation location data 212 with respect to the anatomical map of the heart from heart map data 208. The lesion zone can be presented, for example, as an area or volume of shadow or distinguishing color or shade of color against a surface or area of the representation of the heart. The lesion zone can represent a region of an irreversible electroporation lesion on the heart. After or concurrently with delivery of the pulsed electrical signals to the selected ones of the plurality of electrodes at each of the plurality of locations, the atomical map is automatically annotated on the graphical display by applying an ablation marker based on the predicted lesion zone corresponding to each of the plurality of locations at 308. For example, the anatomical map of the heart can be updated to indicate the region of an irreversible electroporation lesion. In one example, each predicted lesion zone and the corresponding ablation marker have substantially the same visual appearance. In some examples, the anatomical map of the heart can be further updated to indicate lesions of reversible electroporation.

In one example, the controller is further configured to generate, on the anatomical map on the graphical display, a first overlap zone defined by an area of overlap of the corresponding ablation marker and a previously applied ablation marker at each one of the plurality of locations at 310. For example, prior to delivery of the pulsed electrical signals to the selected electrodes, an overlap zone corresponding to an intersection of the predicted lesion zone and a previously applied ablation marker is generated on an anatomical map of the heart on a graphical display. After or concurrently with delivery of the pulsed electrical signals to the selected electrodes, the anatomical map is automatically annotated on the graphical display by defining the overlap zone. Further, the controller can generate, on the anatomical map on the graphical display, a second overlap zone defined by an area of overlap of the corresponding ablation marker and two or more previously applied ablation marker at each one of the plurality of locations. For example, the first overlap zone and the second overlap zone each have a different visual appearance on the anatomical map, such as different shade of a color or a different color. For instance, the first overlap zone has an appearance of a first shade of a color and the second overlap zone has an appearance of a second shade of the color. The first shade of the color may be lighter than the second shade of the color. The controller can be configured to automatically annotate the anatomical map on the graphical display to identify each ablation marker that overlaps with at least two different ablation markers. In another example, the controller is further configured to automatically annotate the anatomical map on the graphical display to display an outer boundary of a series of ablation markers that each overlap with at least two spatially-adjacent ablation markers. For instance, the outer boundary of the series of ablation markers that each overlap may be a line of a different color or a different shade than the color of the overlap zones. The controller can be configured to automatically identify a gap between any two spatially-adjacent ablation markers that overlap with at least two different ablation markers, in which the gap represents an area of the ablation where does not include an overlap zone.

In one example, process 300 can be implemented as set of processor-executable instructions, such as instructions 206, stored in a non-transitory memory, such as memory 204 to be executed by a processor 202 to configure controller 200. The instructions to implement process 300 can be configured to receive information, such as to retrieve from memory 204, heart map data 208, ablation parameter data 210, and ablation location data 212. Further, the instructions to implement process 300 can be configured to annotate, adjust, or write to heart map data 208 and to generate a visualization, such as visualization 220 on a display of graphical representations.

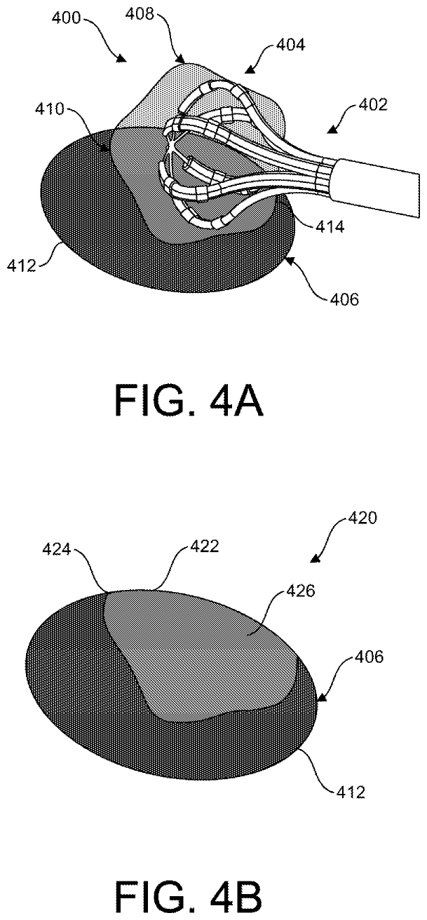

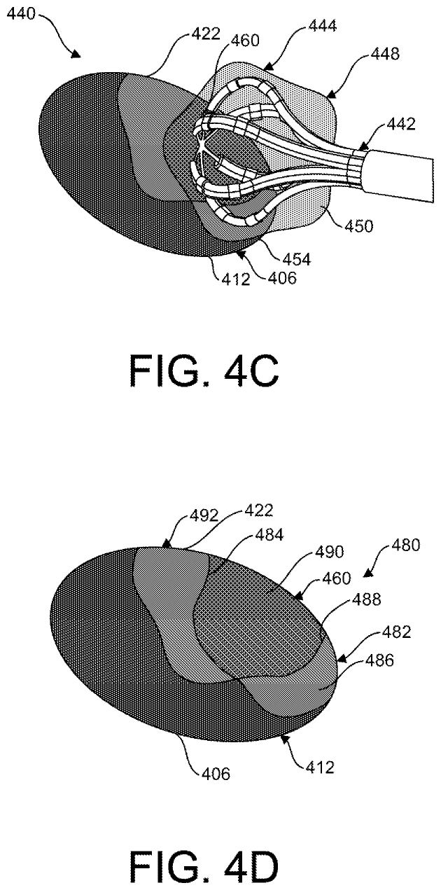

FIG. 4A illustrates a first example graphical representation 400 such as a visualization on a display, such as display 92, as can be configured by controller 200 with electrophysiology system 50 of FIG. 1 and implemented with process 300. In the electrophysiology system, an electroporation catheter 105 has been positioned such that a corresponding electrode assembly is proximate target tissue, such as a surface of a chamber of a patient's heart. As illustrated in FIG. 4A, the first example graphical representation 400 includes a first graphical representation of the catheter 402, a first graphical representation of the electrode assembly 404, and a graphical representation of the target tissue 406, such as the surface of the target tissue, from the anatomical map. Additionally, the first example graphical representation 400 includes a graphical representation of a first model electric field 408 generated with the electrode assembly proximate the target tissue. In one example, a user of controller 200 can select a resolution of the first model electric field 408 for the graphical representation. For instance, the extent or size of the first model electric field 408 can be based on a threshold value of electric field strength, such as the area of the electric field likely capable of producing irreversible electroporation in the target tissue. In one example, the first model electric field 408 can indicate the range of the electric field having an electric field strength of 400 V/cm. Additionally, the model electric field can be indicated with selected visual transparency so that the clinician may observe proximate target tissue. In other examples, the visual appearance of model electric field can indicate an alert such as if the field strength is not sufficient to provide irreversible electroporation or if the field strength is extreme or past a threshold upper limit.

In the example of FIG. 4A, the catheter is configured in a first mode, such as a monopolar mode although aspects of this disclosure are irrespective of mode. During monopolar operation, an electrode, a group of electrodes, or the entire electrode assembly are configured as one of an anode or a cathode, and none of the electrodes in the electrode assembly are configured as a the other of the cathode or the anode. An electrical field is generated between the activated electrodes and a pad dispersive electrode via an electrical signal provided to the catheter, such as pulsed electrical signal. The implementation of the electric field in monopolar mode based on the pulsed signal with respect to the target tissue may be of interest to a clinician. The first model of the electric field 408 based on the pulsed signal and the selected electrode configuration in monopolar mode is generated on the first example graphical representation 400. The visual appearance of the first model electric field 408 can also be based on mode, such as a first color representing the electrode array configured in a monopolar mode and a second color representing the electrode array configured in a bipolar mode. In one example of the controller 200 generating the first example graphical representation 400, the model electric field can be determined from the ablation parameter data 210.

In one example, the position of the first model electric field 408 with respect to the graphical representation of the target tissue 406 and the first graphical representation of the catheter 402 can change as the clinician manipulates the catheter 105 with respect to the heart 30 as well as configures the settings on the electroporation console 130. For instance, as the clinician manipulates, or moves the catheter 105 with respect to the heart 30, the graphical representations, such as the first example graphical representation 400 can track the corresponding movement with the position of the first graphical representation of the catheter 402, first graphical representation of the electrode assembly 404, and first model electric field 408 with respect to the graphical representation of the target tissue 406. Also, as the clinician varies the settings of the electroporation console 130, the corresponding adjustments can be reflected in the geometry, such as size and shape, of the model electric field 408 with respect to the first graphical representation of the catheter 402 and the graphical representation of the target tissue 406, which can provide for titration feedback regarding therapy.

The first example graphical representation 400 includes a lesion zone 410 at the intersection of the graphical representation of the surface of the target tissue 406 and the first model electric field 408. In the example, the first lesion zone 410 presents a graphical representation of the size and location, with respect to the target tissue, of an area (or volume) of the target tissue subjected to irreversible electroporation. In one example, the first lesion zone 410 can include a visual appearance distinguishing the first lesion zone 410 from the first model electric field 408 and unablated regions of target tissue 412.

During manipulation of the electroporation catheter 105 and prior to delivery of pulsed signals to the electrode assembly to effect electroporation, the first lesion zone 410 is a first predicted lesion 414 that provides a visualization as to the size and location, with respect to the target tissue, of a lesion, such as a lesion via irreversible electroporation, created with the current settings of the electroporation catheter system 60 and the current position of the catheter with respect to the heart 30. Prior to the delivery of pulsed electrical signals to the electrode assembly to effect electroporation, the first predicted lesion zone 414 can move or the geometry can change on the graphical representation of the target tissue 406 and with respect to other features of the first example graphical representation 400 as the catheter 105 correspondingly moves with respect to the heart 30 or as the settings of the electroporation console 130 are adjusted. For example, the first model electric field 408 and corresponding first predicted lesion zone 414 can move and change geometry based on a change in the shape of an expandable or flexible group of electrodes or a group of electrode selected to be activated, such as a single spline configuration of activated electrodes, a double spline configuration, or a tip-only configuration.

FIG. 4B illustrates a second example graphical representation 420 such as a visualization on a display, such as display 92, that can be configured by controller 200 with electrophysiology system 50 and implemented with process 300. For instance, the second example graphical representation 420 can be at a time after the delivery of a pulsed electrical signal to the electrodes to affect an ablation of the target tissue in the region corresponding with the first predicted lesion zone 414 on the graphical representation of target tissue 406 of the first example graphical representation 400 in FIG. 4A. The electrode assembly of the electroporation catheter 105 is not proximate the region of the heart corresponding with the graphical representation of the target tissue 406 such that the second example graphical representation 420 does not include a graphical representation of the catheter. The second example graphical representation 420 includes an ablation marker 422 in place of the first predicted lesion zone 414.

After or concurrently with the delivery of the pulsed electrical signal to the electrodes at the location of the electrode assembly proximate the target tissue, the graphical representation of the target tissue 406 is annotated by applying an ablation marker 422 based on the first predicted lesion zone 414. The ablation marker 422 can represent an area on the surface of the patient's heart 30 or a volume of the patient's heart tissue that has been irreversibly electroporated. The ablation marker 422 can be characterized as having an ablation boundary 424 delineating an ablation zone 426 corresponding with an area of the surface of the patient's heart that has been ablated by irreversible electroporation adjacent to unablated regions of target tissue 412. The graphical representation of the target tissue 406 is annotated such that the ablation marker 422 is made a fixture of the anatomical map, and the ablation marker 422 is evident and applied to the geometric anatomical map and as well as applied to and the effects of the irreversible electroporation are present on the electroanatomical map. In one example, the first predicted lesion zone 414 and the corresponding ablation marker 422 can have substantially the same geometry or the same visual appearance. In another example, the first predicted lesion zone 414 can include a visual appearance distinguishable from the visual appearance of the corresponding ablation marker 422 or other ablation markers.

FIG. 4C illustrates a third example graphical representation 440 such as a visualization on a display, such as display 92, that can be configured by controller 200 with electrophysiology system 50 and implemented with process 300. For instance, the third example graphical representation 440 can be at a time after the second example graphical representation 420 of FIG. 4B. The third example graphical representation 440 includes the graphical region of target tissue 406 including unablated tissue 412 and the ablation marker 422 representing a region of ablated tissue. The electroporation catheter 105 has been repositioned such that the corresponding electrode assembly is proximate target tissue but at a location that is offset from the location presented in the first example graphical representation 400 illustrated in FIG. 4A. Accordingly, the third example graphical representation 440 includes a second graphical representation of the catheter 442, a second graphical representation of the electrode assembly 444 and a second model electric field 448. For ease of illustration, the ablation parameter data 210 used to model the third graphical representation 440 is substantially similar to the first graphical representation 400, including the monopolar mode in which the catheter is configured.

In one example, the position of the second model electric field 448 with respect to the graphical representation of the target tissue 406 and the second graphical representation of the catheter 442 can change as the clinician manipulates the catheter 105 with respect to the heart 30 as well as configures the settings on the electroporation console 130. Also, as the clinician varies the settings of the electroporation console 130, the corresponding adjustments can be reflected in the geometry, such as size and shape, of the model electric field 448 with respect to the second graphical representation of the catheter 442 and the graphical representation of the target tissue 406.

The third example graphical representation 440 includes a lesion zone 450 at the intersection of the graphical representation of the surface of the target tissue 406 and the second model electric field 448. In the example, the second lesion zone 450 presents a graphical representation of the size and location, with respect to the target tissue, of an area (or volume) of the target tissue subjected to irreversible electroporation. In one example, the second lesion zone 450 can include a visual appearance distinguishing the second lesion zone 450 from the second model electric field 448, the ablation marker 422, and unablated regions of target tissue 412. The third example graphical representation 440 also includes an overlap zone 460 where the lesion zone 450 and ablation marker 422 overlap. The overlap zone 460 represents a region of tissue where an electric field having strength to irreversibly electroporate the tissue, as indicated by the second model electric field 448, intersects an area of the target tissue that has previously been ablated, as indicated by the ablation marker 422.

During manipulation of the electroporation catheter 105 and prior to delivery of pulsed signals to the electrode assembly to effect electroporation at the second location, the second lesion zone 450 is a second predicted lesion 454 that provides a visualization as to the size and location, with respect to the target tissue, of a lesion created with the current settings of the electroporation catheter system 60 and the new position of the catheter with respect to the heart 30. Prior to the delivery of pulsed electrical signals to the electrode assembly to effect electroporation, the second predicted lesion zone 454 can move or the geometry can change on the graphical representation of the target tissue 406 and with respect to other features of the third example graphical representation 440 as the catheter 105 correspondingly moves with respect to the heart 30 or as the settings of the electroporation console 130 are adjusted. For example, the second model electric field 448 and corresponding second predicted lesion zone 414 can move and change geometry based on a group of electrodes selected to be activated as well as a clinician manipulating the catheter 105 to generate a selected overlap zone 460.

FIG. 4D illustrates a fourth example graphical representation 480 such as a visualization on a display, such as display 92, that can be configured by controller 200 with electrophysiology system 50 and implemented with process 300. For instance, the fourth example graphical representation 480 can be at a time after the delivery of a pulsed electrical signal to the electrodes to affect an ablation of the target tissue in the region corresponding with the second predicted lesion zone 454 on the graphical representation of target tissue 406 of the third example graphical representation 400 in FIG. 4C. The electrode assembly of the electroporation catheter 105 again is not proximate the region of the heart corresponding with the graphical representation of the target tissue 406 such that the fourth example graphical representation 480 does not include a graphical representation of the catheter. The fourth example graphical representation 480 includes a second ablation marker 482 in place of the second predicted lesion zone 454 and includes ablation marker 422 and unablated tissue 412. The fourth example graphical representation 480 also includes an overlap zone 460 in which a region of ablation marker 482 intersects with ablation marker 422.

After or concurrently with the delivery of the pulsed electrical signal to the electrodes at the second location of the electrode assembly proximate the target tissue, the graphical representation of the target tissue 406 is annotated by applying a second ablation marker 482 based on the second predicted lesion zone 454. The first and second ablation markers 422, 482 can represent an area or areas on the surface of the patient's heart 30 or a volume of the patient's heart tissue that has been irreversibly electroporated. The second ablation marker 482 can be characterized as having an ablation boundary 484 delineating an ablation zone 486 corresponding with an area of the surface of the patient's heart that has been ablated by irreversible electroporation adjacent to unablated regions of target tissue 412. The overlap zone 460 can be characterized as having an overlap boundary 488 delineating the overlap zone 460 and an overlap region 490 within the overlap boundary 488 corresponding with an area of the surface of the patient's heart that has been ablated more than once, or subjected to multiple irreversible electroporation, and adjacent to unablated regions of target tissue 412 or tissue that has been ablated only once 492, that is, tissue that has not been subjected to multiple irreversible electroporation.

The graphical representation of the target tissue 406 is annotated such that the ablation marker 482 is made a fixture of the anatomical map in addition to ablation marker 422, and the ablation markers 422, 482 are evident and applied to the geometric anatomical map and as well as applied to and the effects of the irreversible electroporation are present on the electroanatomical map. In one example, the second predicted lesion zone 454 and the corresponding second ablation marker 482 can have substantially the same geometry or the same visual appearance, which can include the same visual appearance as the first ablation marker 422. In another example, the first predicted lesion zone 454 can include a visual appearance distinguishable from the visual appearance of the corresponding ablation marker 482 or other ablation markers such as ablation marker 422. In one example, each ablation marker, such as ablation markers 422, 482 can be indicated on a graphical representation as a selected shade of a selected color, and the overlap zone of two overlapping ablation markers, such as overlap zone 460 can be indicated on the graphical representation as another shade, such as a darker shade, of the selected color. In the case of more than two ablation markers forming an overlap zone representing target tissue that has been subject to more than two overlapping ablations, the overlap zone can be indicated in still another shade, such as an even darker shade, of the selected color. The multiple overlap zones can be defined by an area of overlap of an ablation marker and two or more previously applied overlapping ablation markers. In this example, an overlap zone and the multiple overlap zone each have a different visual appearance on the anatomical map.

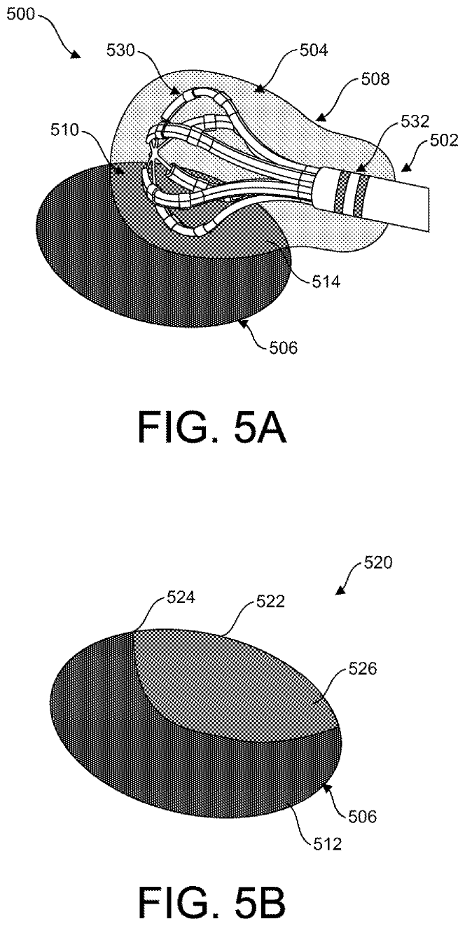

FIG. 5A illustrates another example graphical representation 500 such as a visualization on a display, such as display 92, as can be configured by controller 200 with electrophysiology system 50 and implemented with process 300. In the electrophysiology system, an electroporation catheter 105 has been positioned such that a corresponding electrode assembly is proximate target tissue, such as a surface of a chamber of a patient's heart. In this example, the catheter is configured in a second mode, such as a bipolar mode. During bipolar operation of the catheter 105, a first set of one or more electrodes of the electrode assembly is configured as the anode and a second set of one or more electrodes of the electrode assembly is configured as the cathode to generate the electric field. As illustrated in FIG. 5A, the first example graphical representation 500 includes a graphical representation of the catheter 502, a graphical representation of the electrode assembly 504 configured in bipolar mode, and a graphical representation of the target tissue 506, such as the surface of the target tissue, from the anatomical map. Additionally, the example graphical representation 500 includes a graphical representation of a model electric field 508 generated with the electrode assembly proximate the target tissue. In this example, the electrodes located on the electrode deployment mechanism of the electrode assembly can be configured as an anode or cathode (as represented by first group of electrodes 530), and the catheter includes electrodes located on the catheter shaft (as represented by second group of electrodes 532), which are configured as the other of the cathode and the anode, to generate the electric field.

The example graphical representation 500 includes a lesion zone 510 at the intersection of the graphical representation of the surface of the target tissue 506 and the model electric field 508. In the example, the lesion zone 510 presents a graphical representation of the size and location, with respect to the target tissue, of an area (or volume) of the target tissue that can be or has been subjected to irreversible electroporation. During manipulation of the electroporation catheter 105 in bipolar mode and prior to delivery of pulsed signals to the electrode assembly to effect electroporation, the lesion zone 510 is a predicted lesion 514 that provides a visualization as to the size and location, with respect to the target tissue, of a lesion, such as a lesion via irreversible electroporation, created with the current settings of the electroporation catheter system 60 in bipolar mode and the current position of the catheter with respect to the heart 30.

In some implementations, the electroporation catheter system 60 can selectively toggle between a monopolar mode and a bipolar mode. The implementation of the electric field in monopolar mode based on the pulsed signal with respect to the target tissue versus an implementation of the electric field in a bipolar mode may be of interest to a clinician. For instance, the geometry of the predicted lesion created by a catheter in monopolar mode may differ from the geometry of a predicted lesion created by the catheter in bipolar mode. In one example, a model electric field in a first mode, such as model electric field 508 in bipolar mode, and predicted lesion created with the electric field in the first mode, such as predicted lesion 514 in bipolar mode, can be represented in with different indicia than a model electric field and predicted lesion, respectively, in a second mode, such as monopolar mode. In one example, the model electric field and corresponding predicted lesion in a first mode can be indicated in a first color and the model electric field and corresponding predicted lesion in a second mode can be indicated in a second color. Additionally, portions of the model electric field created by a catheter in bipolar mode, such as portions of the field proximate the catheter shaft, may be undesirable for irreversible electroporation. Selected portions of the model electric field can be indicated in one color, such as a color to indicate a more desirable model electric field, and other selected portion can be indicated in another color, such as a color to indicate a less desirable model electric field.

FIG. 5B illustrates a still another example graphical representation 520 such as a visualization on a display, such as display 92, that can be configured by controller 200 with electrophysiology system 50 and implemented with process 300. For instance, the still another example graphical representation 520 can be at a time after the delivery of a pulsed electrical signal to the electrodes to affect an ablation of the target tissue in the region corresponding with the predicted lesion zone 514 on the graphical representation of target tissue 506 of the first example graphical representation 500 in FIG. 5A. The still another example graphical representation 520 includes an ablation marker 522 in place of the first predicted lesion zone 514. In this example, the geometry of the ablation marker 522 of a lesion created with the catheter in bipolar mode is different than a geometry of an ablation marker of a lesion created with the catheter in monopolar mode.

After or concurrently with the delivery of the pulsed electrical signal to the electrodes at the location of the electrode assembly proximate the target tissue, the graphical representation of the target tissue 506 is annotated by applying an ablation marker 522 based on the first predicted lesion zone 514. The ablation marker 522 can represent an area on the surface of the patient's heart 30 or a volume of the patient's heart tissue that has been irreversibly electroporated. The ablation marker 522 can be characterized as having an ablation boundary 524 delineating an ablation zone 526 corresponding with an area of the surface of the patient's heart that has been ablated by irreversible electroporation adjacent to unablated regions of target tissue 512.

In the examples above, the predicted lesion zone or ablation markers can be presented on the graphical representations as either indicia indicating geometry of the irreversible electroporation on the surface of structures on the anatomical map or as markers indicating a geometry of the irreversible electroporation on the surface as well as indicating depth and volume into the tissue. In the latter, the predicted lesion zone or ablation marker can be a three-dimensional manifestation on the presented graphical representation. Depth can be adjusted in the case of overlap zones based on an understanding of depth effects of repeated ablations.

The above examples describe configurations of a controller to place ablation markers to indicate regions of the tissue subjected to irreversible ablation. In some examples, the controller can be configured to place markers to indicate regions that have received the effects of electrical fields not strong enough to create irreversible electroporation. For example, selected indicia can be applied to annotate regions of reversible electroporation or regions that received therapy not strong enough to create reversible electroporation. In addition, the controller can be configured to apply annotations to regions without the concurrent or subsequent delivery of pulsed electrical signals to the electrode. In one example, areas that can be annotated without associated ablation can include structures or areas around structures such as the sinoatrial node or atrioventricular node such as to provide indications to avoid performing ablation in such regions of the heart. In some examples, the annotation may be temporary, such as for regions of reversible electroporation or regions without associated ablation.

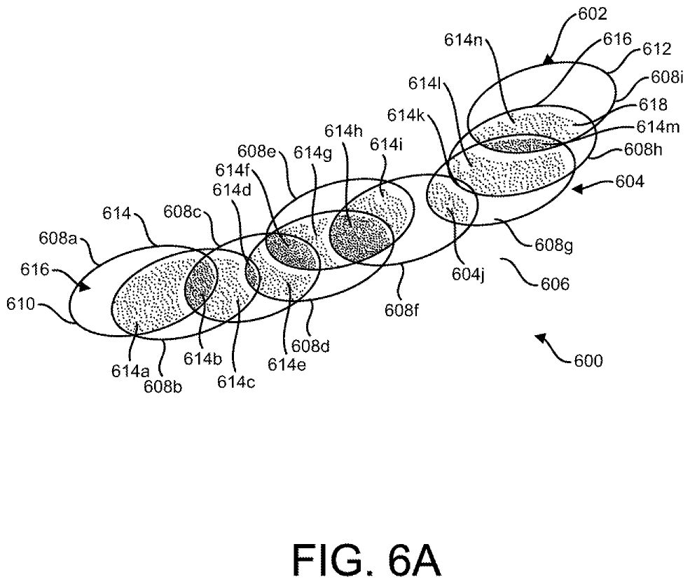

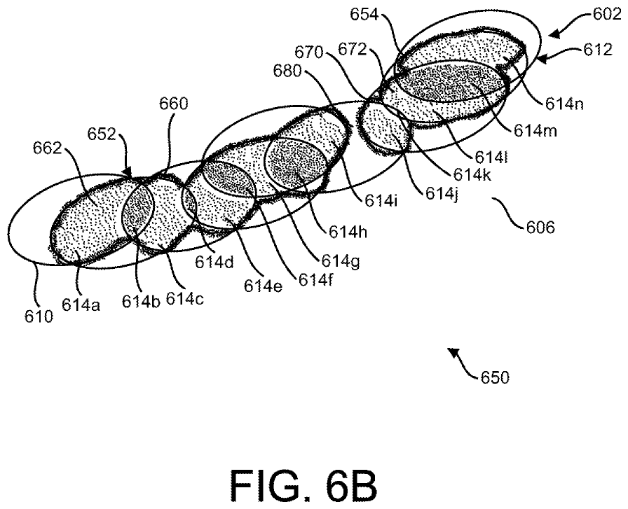

FIG. 6A illustrates an example graphical representation 600 of ablated target tissue in a visualization on a display, such as display 92, that can be configured by controller 200 with electrophysiology system 50 and implemented with process 300. The graphical representation 600 of the ablated target tissue includes lesion line 602 representing irreversibly electroporated tissue 604 proximate unablated tissue 606. The lesion line 602 is defined by a plurality of ablation markers 608a-608i generated, for example, in the manner described in the disclosure, providing a visual indication to the clinician of the overall state of the ablation procedure, e.g., to assess the likelihood that a complete conduction block has been achieved. In this example, the first ablation marker 608a represents a first end 610 of the lesion line 602, and the ninth ablation marker 608i represents a second end 612 of the lesion line 602. It will be readily understood, however, that the specific number and positions of individual ablation markers can vary in a given procedure. Additionally, in the illustrated example, the individual ablation markers 608a-608i are depicted as being generally elliptical in shape. However, this elliptical shape is merely for ease of illustration for purposes of the present disclosure. That is, the ablation markers 608a-608i can take on any numbers of shapes and appearances, as discussed elsewhere herein.

As explained above, a lesion line, such as lesion line 602, represents a length of irreversibly electroporated tissue contiguously from the first end to the second end and includes multiple ablation markers. The contiguously connected ablation markers 608a-608i can be ablation markers that are touch each other or ablation markers that include a region of overlap. The lesion line 602 can be characterized as having at least two spatially-adjacent ablation markers forming a contiguous lesion line boundary 614 adjacent to unablated tissue (represented by element 606 in FIG. 6A), as an outer limits of the lesion line 602, delineating a lesion region 616 of ablated tissue 604 within the lesion line boundary 614.

In one example, the controller, such as controller 200 of FIG. 2, can be configured to automatically identify and annotate a lesion line, such as lesion line 602, and implemented with process 300 at 310 of FIG. 3. The controller can determine whether an ablation marker is spatially-adjacent to another ablation marker to form a lesion line and automatically annotate the lesion line on a graphical representation. For example, the controller can be configured to automatically highlight the contiguous lesion line boundary 614 with visual indica or to automatically highlight with distinguishing shades or colors the spatially-adjacent ablation markers on a graphical representation. In one example, the lesion line is automatically identified in real-time as the graphical representation presents two or more spatially-adjacent ablation markers. In another example, the lesion line can be automatically identified at a selected time via controls. Automatic identification of the lesion line provides feedback to the clinician as to whether multiple nearby ablation markers are separated by unablated tissue. In one example, the controller can be configured to automatically highlight a space of unablated tissue disposed between multiple ablation markers, such as with indicia to provide a visual alert to the user.

The lesion line 602 of the example also includes a plurality of overlap zones 614, which represents areas of tissue that have overlapping regions of ablation, such as tissue that has been subjected to more than one irreversible electroporation step, which can provide the clinician with a visual indicia of the likelihood that a complete, transmural lesion has been created. For example, the first ablation marker 608a and the second ablation marker 608b intersect in a region of the target tissue as illustrated to form a first overlap zone 614a. Also, the first ablation marker 608a, second ablation marker 608b, and third ablation marker 608c intersect as illustrated to form a second overlap zone 614b. The illustrated first overlap zone 614a is a two-marker overlap zone as the first overlap zone 614a is formed of two applications of intersecting irreversible electroporation to the same area of tissue. The illustrated second overlap zone 614b is a three-marker overlap zone as the second overlap zone 614b is formed of three applications of intersecting irreversible electroporation to the same area of tissue. Overlap zones of more than three applications of intersecting irreversible electroporation to an area of tissue are also contemplated. The nine ablation markers 608a-608i of the lesion line 602 form fourteen overlap zones 614a-614n as illustrated. Each overlap zone 614a-614n, such as overlap zone 614n for example, can be characterized as having an overlap boundary 616 delineating the overlap zone, such as 614n, and an overlap region 618 within the overlap boundary 616.

FIG. 6B illustrates an example graphical representation 650 of ablated target tissue in a visualization on a display, such as display 92, that can be configured by controller 200 with electrophysiology system 50 and implemented with process 300 at 310 highlighting overlap zones, such as overlap zones 614a-614n. Regions of overlapping ablation include a higher probability of a durable lesion over regions of non-overlapping ablation, in which the tissue of interest has only been subjected to a single dose of irreversible electroporation. Thus, a clinician may find it desirable to form and identify a contiguous string of overlap zones in the tissue. In one example, the controller, such as controller 200 of FIG. 2, can be configured to automatically identify and annotate a contiguous string of two or more overlap zones on the graphical representation 650, such as first contiguous string 652 and second contiguous string 654, at 310 of FIG. 3. The first contiguous string 652 of overlap zones includes a plurality of contiguously spatially-adjacent overlap zones, such as overlap zones 614a-614i in which overlap zone 614a is spatially-adjacent to overlap zone 614b, and so on to overlap zone 614i. The second contiguous string 654 of overlap zones includes a plurality of contiguously spatially-adjacent overlap zones, such as overlap zones 614j-614n in which overlap zone 614j is spatially-adjacent to overlap zone 614k, which is spatially-adjacent to overlap zone 614l, which is spatially-adjacent to overlap zone 614m, which is spatially adjacent to overlap zone 614n. The contiguous string 652 can be characterized by a string boundary 660 and a contiguous region 662 of spatially-adjacent overlap zones within string boundary 660. The contiguous string 654 can be characterized by a string boundary 670 and a contiguous region 672 of spatially-adjacent overlap zones within string boundary 670.

The controller can determine whether an overlap zone is spatially adjacent to another overlap zone to form a contiguous string, and automatically annotate the contiguous string on a graphical representation. For example, the controller can be configured to automatically highlight the contiguous string with visual indica such as a highlighted string boundary 662, 672, or to automatically highlight with distinguishing shades or colors the spatially-adjacent overlap zones on a graphical representation. In one example, the contiguous string is automatically identified in real-time as the graphical representation presents two or more spatially-adjacent overlap zones. In another example, the contiguous string can be automatically identified at a selected time via controls.

Automatic identification of the contiguous strings 652, 654 provides feedback to the clinician as to whether a lesion line includes a nonoverlap section, such as gap 680 between contiguous strings 652, 654 on the graphical representation 650. In one example, the controller can be configured to automatically highlight the gap 680 such as via a visual alert if, as determined by the controller, the gap 680 may be interesting to a clinician. The controller can be configured to determine whether a gap may be interesting such a determination that the contiguous string and gap are included in a lesion line, whether the distance or centroid distance between overlap zones proximate the gap is within a threshold amount, or other determinations.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for performing electroporation ablation of target tissue in a chamber of a patient's heart, the system comprising:
a catheter including an electrode assembly having a plurality of electrodes, wherein the catheter is adapted to position the electrode assembly at a first location proximate the target tissue;
a graphical display; and
a controller configured to:
prior to delivery of ablative energy to the plurality of electrodes:
generate, on the graphical display, a graphical representation of a model of electric fields generated by the plurality of electrodes; and
generate, on an anatomical map of the heart on the graphical display, a first predicted lesion marker corresponding to an intersection of the model of electric fields and a surface of the anatomical map when the electrode assembly is at a first position proximate the target tissue; and
after or concurrently with delivery of ablative energy to the plurality of electrodes, automatically annotate the anatomical map on the graphical display with a first ablation marker corresponding to the first predicted lesion marker.

2. The system of claim 1, wherein the controller is further configured to, when the electrode assembly is at a second location proximate the target tissue and after automatically annotating the anatomical map with the first ablation marker, generate, on the atomical map of the heart on the graphical display, a second predicted lesion marker corresponding to the intersection of the model of electric fields and the surface of the anatomical map prior to delivery of the ablative energy to the plurality of electrodes.

3. The system of claim 2, wherein the controller is further configured to, after or concurrently with delivery of the ablative energy to the plurality of electrodes when the electrode assembly is at the second position proximate the target tissue, automatically annotate the anatomical map on the graphical display with a second ablation marker corresponding to the second predicted ablation marker.

4. The system of claim 2, wherein the controller is further configured to, when the electrode assembly is at the second position proximate the target tissue and after automatically annotating the anatomical map with the first ablation marker, generate, on the atomical map of the heart on the graphical display, a third predicted lesion marker defined by an area of overlap of the first ablation marker and the second predicted lesion marker.

5. The system of claim 4, wherein the controller is further configured to, after or concurrently with delivery of the ablative energy to the plurality of electrodes when the electrode assembly is at the second position proximate the target tissue, automatically annotate the anatomical map on the graphical display with a third ablation marker corresponding to the third predicted ablation marker.

6. The system of claim 5, wherein the third ablation marker has a different visual appearance than the first and second ablation markers.

7. The system of claim 1, wherein the first predicted lesion marker and the first ablation marker each have a different visual appearance.

8. The system of claim 1, wherein the catheter is configured for selective delivery of monopolar and bipolar ablative energy, and wherein the controller is configured to generate the model of the electric fields when the catheter configured for delivery of monopolar ablative energy differently than when the catheter is configured for delivery of bipolar ablative energy.

9. A system for performing electroporation ablation of target tissue in a chamber of a patient's heart, the system comprising:
a catheter including an electrode assembly having a plurality of electrodes, wherein the catheter is adapted to position the electrode assembly at a location proximate an ablated region of the target tissue;
a graphical display; and
a controller configured to:
generate, on the graphical display, a graphical representation of the electrode assembly and a first ablation marker corresponding with the ablated region;
generate, on the graphical display, a graphical representation of a model of electric fields generated in response to delivery of pulsed electrical signals to selected ones of the plurality of electrodes;
prior to delivery of the pulsed electrical signals to the selected ones of the plurality of electrodes at the location, generate, on an anatomical map of the heart on the graphical display, a predicted lesion zone corresponding to an intersection of the model of electric fields and a surface of the anatomical map and an overlap zone corresponding to an intersection of the predicted lesion zone and the first ablation marker; and
after or concurrently with delivery of the pulsed electrical signals to the selected ones of the plurality of electrodes at the location, automatically annotate the anatomical map on the graphical display by applying a second ablation marker based on the predicted lesion zone corresponding to the location and by defining the overlap zone.

10. The system of claim 9, wherein the controller is further configured to automatically identify on the anatomical map on the graphical display another overlap zone spatially adjacent to the overlap zone.

11. The system of claim 9, wherein the controller is further configured to automatically annotate on the graphical display a first contiguous string of spatially-adjacent overlap zones including the overlap zone.

12. The system of claim 11, wherein the controller is further configured to automatically annotate on the graphical display a second contiguous string of spatially-adjacent overlap zones spaced-apart on the target tissue from the first contiguous string of spatially-adjacent overlap zones.

13. The system of claim 12, wherein the controller is further configured to automatically identify on the graphical display a gap on the target tissue disposed between the first contiguous string of spatially-adjacent overlap zones spaced-apart and the second contiguous string of spatially-adjacent overlap zones.

14. The system of claim 13, wherein the controller is further configured to automatically highlight on the graphical display the gab based on distance between the first contiguous string of spatially-adjacent overlap zones spaced-apart and the second contiguous string of spatially-adjacent overlap zones.

15. A process for use with electroporation ablation of target tissue in a chamber of a patient's heart with a catheter including an electrode assembly having a plurality of electrodes, wherein the catheter is adapted to position the electrode assembly at a plurality of locations proximate the target tissue, the process comprising:
generating, on a graphical display, a graphical representation of the electrode assembly;
generating, on the graphical display, a graphical representation of a model of electric fields generated in response to delivery of pulsed electrical signals to selected ones of the plurality of electrodes;
prior to delivery of the pulsed electrical signals to the selected ones of the plurality of electrodes at each of the plurality of locations, generating, on an anatomical map of the heart on the graphical display, a predicted lesion zone corresponding to an intersection of the model of electric fields and a surface of the anatomical map; and
after or concurrently with delivery of the pulsed electrical signals to the selected ones of the plurality of electrodes at each of the plurality of locations, automatically annotating the anatomical map on the graphical display by applying an ablation marker based on the predicted lesion zone corresponding to each of the plurality of locations.

16. The process of claim 15, and further generating, on the anatomical map on the graphical display, a first overlap zone defined by an area of overlap of the corresponding ablation marker and one previously applied ablation marker at each of the plurality of locations.

17. The process of claim 16, and further generating, on the anatomical map on the graphical display, a second overlap zone defined by an area of overlap of the corresponding ablation marker and two or more previously applied ablation markers at each of the plurality of locations.

18. The process of claim 16, and further automatically annotating the anatomical map on the graphical display to identify each ablation marker that overlaps with at least two different ablation markers.

19. The process of claim 15, and further automatically identifying on the anatomical map on the graphical display a lesion line of contiguous series of spatially-adjacent ablation markers including the ablation marker and previously applied ablation markers.

20. The process of claim 15, wherein the catheter is configured for selective delivery of monopolar and bipolar ablative energy, generating indicia in the model of the electric fields on the graphical display when the catheter configured for delivery of monopolar ablative energy differently than when the catheter is configured for delivery of bipolar ablative energy.
